(19) United States
(12) Patent Application Publication
(10) Pub. No.: US 2008/0154378 A1
(43) Pub. Date: Jun. 26, 2008

(54) BONE IMPLANT HAVING ENGINEERED SURFACES

(75) Inventor: Mark J. Pelo, Macy, IN (US)

Correspondence Address: HAYNES AND BOONE, LLP, 901 Main Street, Suite 3100, Dallas, TX, 75202, US

(73) Assignee: WARSAW ORTHOPEDIC, INC., Warsaw, IN (US)

(21) Appl. No.: 11/615,158

(22) Filed: Dec. 22, 2006

(51) Int. Cl. A61F 2/44

(52) U.S. Cl. 623/17.16; 623/17.11

(57) ABSTRACT

The present disclosure is directed to an implantable device that is machined to have a bone engaging interface defining cavities that may be deposited with bone growth promoting material.

### BACKGROUND

Severe back pain and nerve damage may be caused by injured, degraded, or diseased spinal joints and, particularly, spinal discs. Similarly, hip and knee pain can be caused by injured, degraded, or diseased hip and knee joints. For example, disc deterioration and other spinal deterioration may cause spinal stenosis, a narrowing of the spinal canal and/or the intervertebral foramen, that causes pinching of the spinal cord and associated nerves. Severe hip joint degradation, for example, can often require implantation of a hip implant in what is commonly referred to as a hip replacement surgery.

Current methods of treating damaged spinal discs include vertebral fusion, nucleus replacements, or motion preservation prostheses. A spinal prostheses joint, such as that described U.S. Pat. No. 6,740,118, the disclosure of which is incorporated herein by reference, for example, is placed between two vertebral bodies to maintain or restore motion similar to the normal motion provided by natural intervertebral joints. Artificial disc implants, such as described in U.S. Pat. No. 6,402,785, the disclosure of which is incorporated herein by reference, have also been used as a disc replacement therapy. Other spinal therapies include fixation systems whereby bone screws, for example, are inserted into vertebral bodies and a connecting rod is secured between the screws to provide spinal stability, such as that described in U.S. Pat. No. 6,454,773, the disclosure of which is incorporated herein by reference.

Generally, surfaces of these implant and other bone-related implant devices are roughened and coated with a bone-growth promoting material, such as Infuse®, which is commercially available from Medtronic, Inc. of Minneapolis, Minn., hydroxyapatite, or other similar bone-growth promoting material. INFUSE is a registered trademark of Medronic Sofamor Danek, Inc, Minneapolis, Minn. Chemical etching, plasma spraying, and porous coating are typically used to roughen the bone engaging surfaces of the implants. With conventional roughening techniques, the roughened surface is randomly patterned. As a result, there is little control in defining the surface pattern or bone engaging interface. Therefore, there is a need for bone implants with engineered surfaces to provide controlled bone growth interfaces.

### SUMMARY

In one aspect, this disclosure is directed to an implant having a body and a bone engaging interface. The bone engaging interface is formed on a portion of the body and is shaped to favor movement of the implant in a first direction and to resist movement in a second direction opposite the first direction.

In another aspect, this disclosure is directed to an intervertebral prosthetic joint that has a first articular component and a second articular component. A first bone engaging surface is defined on a portion of the first articular component and a second bone engaging surface is defined on a portion of the second articular component. Each bone engaging surface provides a migration promoting interface along a first direction and provides an anti-migratory interface along a second direction opposite the first direction.

According to another aspect, this disclosure is directed to an artificial implant having an implant body that includes a bone engaging interface. Cavities are formed by laser machining a portion of the bone engaging surface of the implant body. Bone growth material is then deposited in the cavities.

In yet another aspect, this disclosure is directed to a surgical method for positioning an intervertebral implant. The method includes preparing a disc space for reception of an intervertebral implant. An intervertebral implant is inserted along a first direction into the disc space. The intervertebral implant is then withdrawn from the disc space generally along a second direction opposite the first direction. The implant is withdrawn from the disc space until the implant engages a vertebral body defining the disc space.

This disclosure is also directed to a method of manufacturing an implant. The manufacturing process includes the formation of an implant body. The implant body is laser machined to define a plurality of protrusions and cavities. Bone growth promoting material is deposited into the cavities.

In another aspect, this disclosure is directed to a bone screw. The bone screw has a shaft and a plurality of threads formed thereon. A bone ingrowth cavity is formed in the shaft in a space between a pair of threads.

These and other aspects, forms, objects, features, and benefits of the present invention will become apparent from the following detailed drawings and descriptions.

### BRIEF DESCRIPTION OF THE DRAWINGS

FIG. 1 is a perspective view of an intervertebral prosthetic joint according to one aspect of the present disclosure.

FIG. 2 is a plan view of a portion of the intervertebral prosthetic joint shown in FIG. 1.

FIG. 3 is a cross-sectional view of a portion of that shown in FIG. 2 taken along lines 3-3.

FIG. 4 is a cross-sectional view of a portion of a bone engaging interface according to another example of the present disclosure.

FIG. 5 is a cross-sectional view of a portion of a bone engaging interface according to yet another example of the present disclosure.

FIG. 6 is a cross-sectional view of a portion of a bone engaging interface according to a further example of the present disclosure.

FIG. 7 is a perspective view of a portion of a bone engaging interface according to yet another example of the present disclosure.

FIG. 8 is an elevation view of a bone screw according to one example of the present disclosure.

FIG. 9 is an exploded view of a portion of the bone screw of FIG. 8.

### DETAILED DESCRIPTION

The present disclosure relates generally to the field of orthopedic surgery, and more particularly to systems and methods for replacing or stabilizing a spinal joint. For the purposes of promoting an understanding of the principles of the invention, reference will now be made to embodiments or examples illustrated in the drawings, and specific language will be used to describe the same. It will nevertheless be understood that no limitation of the scope of the invention is thereby intended. Any alteration and further modifications in the described embodiments, and any further applications of the principles of the invention as described herein are contemplated as would normally occur to one skilled in the art to which the disclosure relates. While the present disclosure will be described with respect to spinal or vertebral implants, it is understood that the present disclosure is also applicable with other implant types, such as hip or knee implants.

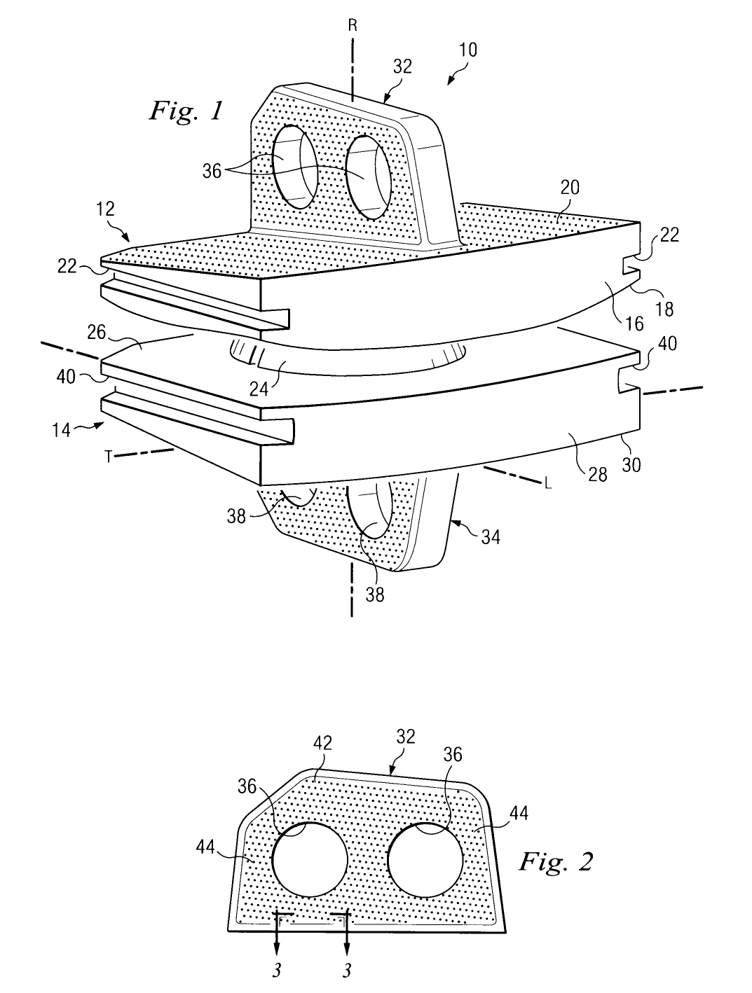

Referring to FIG. 1, a intervertebral prosthetic joint incorporating aspects of the present disclosure is shown. The intervertebral prosthetic joint is similar to that described in U.S. Pat. No. 6,740,118, the disclosure of which is incorporated herein by reference. Joint 10 has two articular components 12, 14 that cooperate to provide rotational and pivotal movement between vertebral members. The joint 10 is designed to simulate the bio-mechanical motion provided by a natural intervertebral disc.

The articular components 12, 14 are permitted to pivot relative to one another about a number of axes, including lateral or side-to-side pivotal movement about longitudinal axis L and anterior-posterior pivotal movement about a transverse axis T. In a preferred embodiment of the invention, the articular components 12, 14 are permitted to pivot relative to one another about any axes that lies in a plane that intersects longitudinal axis L and transverse axis T. Additionally, the articular components 12, 14 are preferably permitted to rotate relative to one another about a rotational axis R. Although the articulating joint 10 has been illustrated and described as providing a specific combination of articulating motion, it should be understood that other combinations of articulating movement are also possible and are contemplated as falling within the scope of the present invention. It should also be understood that other types of articulating movement are also contemplated, such as, for example, relative translational or linear motion.

Although the articular components 12, 14 of prosthetic joint 10 may be formed from a wide variety of materials, in one embodiment of the invention, the articular components 12, 14 are formed of a cobalt-chrome-molybdenum metallic alloy (ASTM F799 or F-75). However, in alternative embodiments of the invention, the articular components 12, 14 may be formed of other bio-compatible metallic materials such as titanium or stainless steel, a bio-compatible polymeric material such as polyethylene, or any other biocompatible material that would be apparent to one of ordinary skill in the art. The surfaces of the articular components 12, 14 that are positioned in direct contact with vertebral bone are preferably coated with a bone-growth promoting substance, such as, for example, a hydroxyapatite coating formed of calcium phosphate. Additionally, the surface of the articular components 12, 14 that are positioned in direct contact with vertebral bone include bone growth promoting interfaces, which will be described in greater detail below.

Articular component 12 includes a support plate 16 having an articular surface 18 and an opposite bearing or bone engaging surface 20. Support plate 16 is sized and shaped to substantially correspond to the size and shape of an end plate of an adjacent vertebra (not shown). The articular component 12 also includes a tool engaging groove 22 defined between the articular surface 18 and the bone engaging surface 20, and is designed to receive a tool or other instrument to aid in the placement of the joint between vertebral members.

Articular component 12 includes a concave recess (not shown) formed in the convex articular surface 18 of support plate 16. Preferably, the concave recess has a semi-spherical shape sized to receive a correspondingly shaped projection 24 of articular component 14. Projection 24 extends from substantially planar articular surface 26 of support plate 28 of articular component 14. Opposite of articular surface 26 is bearing or bone engaging surface 30. Similar to articular component 12, a groove 32 designed to receive a corresponding portion of a surgical tool or instrument is formed between the articular surface 26 and bone engaging surface 30 in support plate 28. Extending from the respective bone engaging surfaces 20, 30 of articular components 12, 14 are flange members or keels 32, 34. The keels are sized to fit within an opening formed in adjacent vertebral endplates (not shown). The keels preferably extend perpendicularly from the bone engaging surfaces and are centrally disposed so as to divide the respective bone engaging surfaces in half. Each keel preferably includes a pair of openings 36, 38 to facilitate bone-through growth to promote fixation to adjacent vertebra. While only two openings are shown, it is contemplated that the keels may be constructed to have any number of openings. Additionally, while only a single respective keel is shown extending from the bone engaging surfaces, it is contemplated that the joint may be constructed to have any number of keels, and those keels can be of different shapes and/or sizes.

Articular component 14 also includes a tool engaging groove 40 similar to that of articular component 12. Groove 40 is designed to receive a tool or other instrument to aid in the placement of the joint between vertebral bodies.

As referenced above, the bone engaging surfaces of the articular components provide a bone engaging interface that can be deposited with bone growth promoting or cellular material. In a preferred example, the bone engaging surfaces are machined, using a laser, for instance, to provide a controlled interface that, for example, can include cavities, recesses, grooves, and the like for housing seeds of bone growth promoting material. In another example, the bone engaging interfaces can be formed to favor movement of the joint in one direction but resist movement of the joint in an opposite direction. Laser machining the bone engaging surfaces of the joint provides a textured surface that, unlike chemical etching or plasma spraying, for example, is not necessarily random and thus can be used to develop a pre-defined bone engaging interface. In one example, a pulsing Nd:YV04 laser is used to machine the bone engaging surfaces of the joint. However, it is recognized that other lasers may be used. It also recognized that the bone engaging surfaces may be machined using Electrical Discharge Machining (EDM) or other machining techniques.

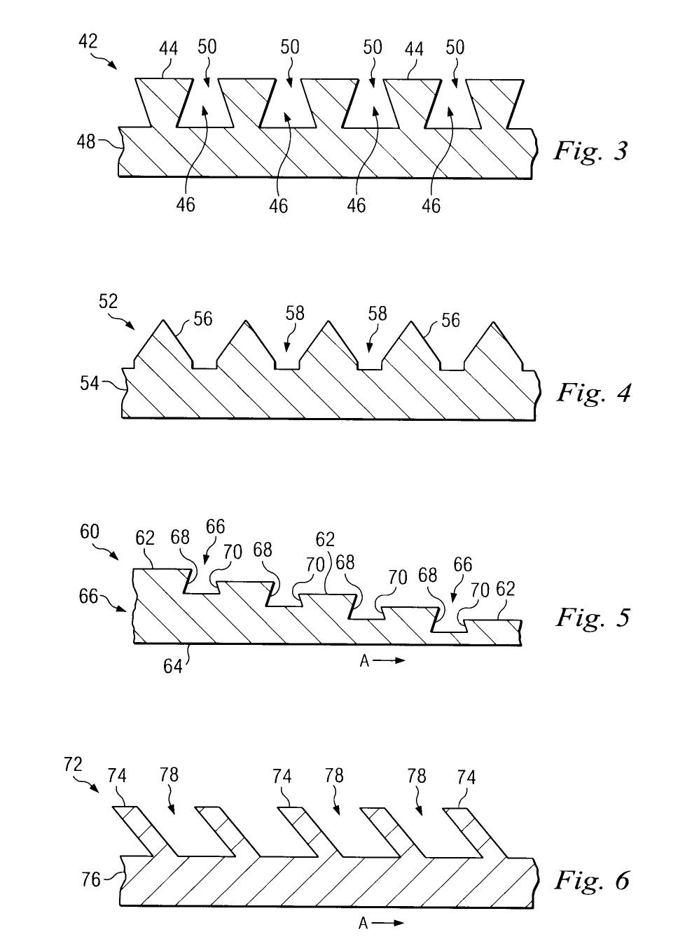

FIG. 2 is an exploded view of keel 32 of articular component 12. As illustrated, the outer surface 42 of the keel 32 has been machined, preferably with a pulsing Nd:YV04 laser, to define a plurality of inverted frustoconical protrusions 44. As shown in the partial cross-sectional view of FIG. 3, the protrusions 44 define cavities 46 that, in one preferred example, can house bone growth promoting material, such as Infuse, or cellular material. The protrusions 44 taper inwardly to the body 48. As a result, the cavities 46 defined between protrusions 44 are configured to receive bone growth from an adjacent vertebral member and provide a bio-locking function. That is, the cavities 46, as defined by the protrusions 44, are wider at the body 48 than at the bone growth openings 50. Thus, bone may enter a cavity 48 via relatively narrow opening 50 and then fill out the cavity as bone growth continues. Protrusions 44 are shown as being uniformly sized, shaped, and spaced, however, it is contemplated that the protrusions can have varying sizes and shapes. The spacing between protrusions may also vary.

In the illustrated example, the protrusions are formed by machining the body 48. As shown, in this example, the protrusions are identically shaped, sized, and spaced. However, it is contemplated that the protrusions may be machined to be non-uniformly shaped, sized, and/or spaced. Moreover, while only a portion of the bone engaging interface of keel 32 is shown, it is recognized that other portions of the bone engaging interface may be differently constructed from that shown in FIG. 3. In this regard, a bone engaging surface may be machined to have multiple and different bone engaging interfaces. It is also recognized that some portions of the bone engaging surface may be machined whereas other portions are roughened using conventional roughening processes or left smooth. Additionally, it is contemplated that one of various machining techniques can be used for forming the protrusions on implant surfaces that are typically too delicate for surface roughening or relatively inaccessible by conventional surface roughening techniques.

Also, while only the bone engaging interface of a keel has been shown and described, it is understood that other bone engaging surfaces of the joint may be machined to form a bone engaging interface similar to that described herein.

FIGS. 4-8 illustrate other representative bone engaging interfaces that may be formed on a keel or other bone engaging surface of joint 10. It is recognized that the present disclosure is not limited to the representative bone engaging interfaces illustrated in FIGS. 4-8. Moreover, the bone engaging interface protrusions are not limited to the shapes, sizes, or orientations described herein.

FIG. 4 is a partial cross-sectional view of a bone engaging interface according to another example of the present disclosure. The bone engaging interface 52 has a body 54 with a number of pointed (cone-shaped) protrusions 56 extending therefrom. The pointed protrusions 56 are spaced from one another to define a number of cavities 58 that are configured to house bone growth promoting or cellular material. The protrusions are shown as being uniformly sized, shaped, and spaced, but it is contemplated that the protrusions can have varying sizes and shapes. The spacing between protrusions may also vary.

Referring now to FIG. 5, a partial cross-sectional view of a bone engaging interface according to another example of the present disclosure is shown. The bone engaging interface 60 differs from the bone engaging interfaces heretofore shown in that the height of protrusions 62 extending from body 64 varies. In the illustrated example, the protrusions 62, which are spaced from one another to define cavities 66, have a “stair-stepped” profile. Cavities 66 are designed to house bone growth promoting or cellular material.

As illustrated, the height of the protrusions 62 increases from left-to-right along the profile of the bone engaging interface 60. In one preferred example, the direction of descending protrusion height, as indicated by arrow A, coincides with the direction of implantation. That is, the bone engaging interface 60 is constructed such that end 64 represents the leading edge of the interface and end 66 represents the trailing edge of the interface. This configuration of the bone engaging interface 60 allows the implant to bite in or otherwise engage the adjacent vertebral member when being implanted. Additionally, when preparing the vertebral member for the keel, for example, a slight taper can be cut into the vertebral member that matches the taper provided by the bone engaging interface 60.

Bone engaging interface 60, as referenced above, is designed to scratch or otherwise bite into the vertebral member when implanted in the vertebral member. Specifically, the trailing walls 68 of protrusions 62 are angled to engage the vertebral member when the implant is inserted into the vertebral member. As a result, cellular material can be scraped from the vertebral member and into the cavities 66 during implantation. This cellular material can then help promote bone growth into the cavities 66. Further, the height and angle of the trailing walls 68 can be controlled during fabrication to provide a desired degree of bio-scraping. In other words, the amount of cellular material scraped from the vertebral member and deposited in cavities 66 can be controlled by precise formation of the bone engaging interface. The leading walls 70 of the bone engaging interface 60 bite into the vertebral member along a direction opposite the direction of implantation. In this regard, when loaded, the bone engaging interface is locked relatively in place.

In the example illustrated in FIG. 5, the height of the protrusions 62 decrease, or descend, in the direction of implantation. However, it is contemplated that the height of the protrusions could increase, or ascend, in the direction of implantation.

FIG. 6 is a cross-sectional view of another representative bone engaging interface according to the present disclosure. In this example, the bone engaging interface 72 has angled protrusions 74 extending from body 76 and defining cavities 78 that, in a preferred example, are configured to house bone growth promoting material or scaped or filed in cellular material. As illustrated, the protrusions 74 are angled in a direction opposite to the direction of implantation, referenced by arrow A. In this regard, bone engaging interface 72 is designed to slide, relatively easily, into the vertebral member without much degradation of the engaging surface of the vertebral member. However, as the protrusions 74 are angled in a direction opposite the direction of implantation, the bone engaging interface 72 is designed to bite into the vertebral member when the bone engaging interface 72 is withdrawn from or otherwise moved in a direction opposite the direction of implantation. Thus, in one preferred method of using an implant having a bone engaging interface similar to that shown in FIG. 6, a surgeon inserts the implant into position along a direction of implantation. To fix the implant into position, the surgeon then begins to withdraw the implant in a direction opposite the direction of implantation. The surgeon continues to withdraw the implant until the angular protrusions bite or cut into the vertebral member. It is recognized that the surgeon may need to slightly angle the implant to assist with the engagement of the bone engaging interface with the vertebral member. Similar to the bone engaging interfaces described above, cavities 78 preferably are deposited with bone growth promoting material, e.g., filed cellular material.

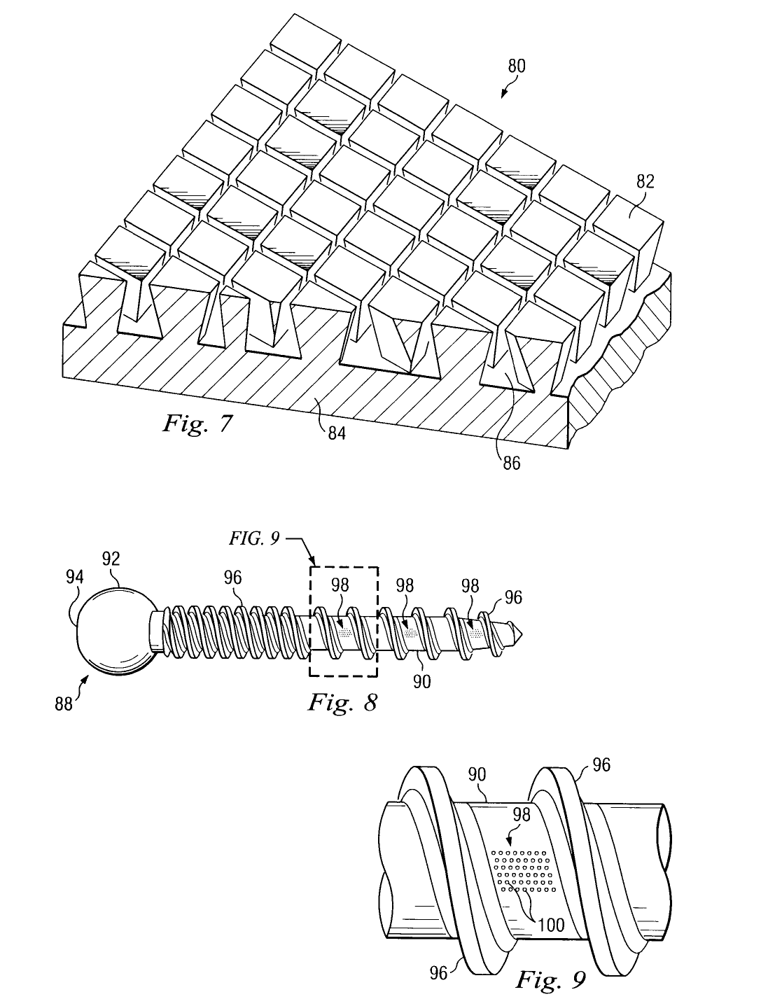

In the example illustrated in FIG. 7, the angled protrusions 74 are angled in a direction opposite to the direction of implantation. However, it is contemplated that the angles could favor, rather than oppose, the direction of implantation.

FIG. 7 is a perspective view of a portion of a bone engaging interface according to another example of the present disclosure. In this example, the bone engaging interface 80 has a series of complex-shaped protrusions 82 extending from body 84. Similar to that described above, the protrusions 82 are spaced from one another to define cavities 86 that preferably house bone growth promoting material and, as shown in FIG. 7, for example, the two-dimensional arrangement of the protrusions defines an in intricate network of cavities that are designed to receive bone growth thereby provide a bio-locking of the implant. Similar to the bone engaging interfaces described above, protrusions 82 are formed by laser machining. Unlike conventional etching techniques, formation of protrusions 82 can be precisely controlled to yield a desired shape, size, and spacing.

The bone engaging interfaces have been described above as being formed on a bone engaging surface of a keel or other member of an articulating prosthetic joint. The bone engaging interfaces have been described as having protrusions that, in one example, are angled to assist with implantation but also provide a bio-locking. In this regard, it is contemplated that the draft of the protrusions can be machined to provide a desired release-ability. That is, with more draft, it would be more difficult to release or remove the implant after bone ingrowth. On the other hand, with less draft, it would be easier for a surgeon to remove the implant after bone ingrowth and, such removal could be done without significant bone loss. Also, it is noted that in the example of FIG. 5 the protrusions are constructed such that the stair-stepped profile descends in the direction of implantation, and in the example of FIG. 6, the protrusions are angled in a direction opposite of the direction of implantation. However, it is contemplated that the protrusions may be constructed such that movement is resisted in directions other than in or opposite the direction of implantation. For example, the implant may be constructed such that the protrusions resist movement in a posterior or anterior direction when the implant is inserted along a lateral approach. Additionally, embodiments of the present disclosure have been described with respect to resisting migration or movement in only one direction; however, it is noted that the implant may be constructed to resist movement in more than direction.

Heretofore, the present disclosure has been described with respect to joint replacements. The present disclosure, however, is not so limited. The present disclosure can be implemented with other implantable devices, such as a bone screw. A representative bone screw is shown in FIG. 8.

Bone screw 88 includes a shaft 90 connected to a curvate head 92. Curvate head 92 has a centrally disposed notch 94 configured to receive the driving end of driving instrument. Bone screw 88 includes a series of threads 96 formed circumferentially around shaft 90. The screw is configured to sit within a rod-receiver coupler (not shown) designed to hold a stabilization rod. The shaft 90 of bone screw 88 includes, in the illustrate example, three engineered bone engaging interfaces 98. These areas of the shaft 90 are, in the illustrated example, disposed between adjacent threads 96 to define bone growth promoting areas along the bone screw. In the illustrated example, the bone engaging interfaces do not extend circumferentially around the shaft 90; however, it is contemplated that an engineering surface may be formed circumferentially around shaft 90.

FIG. 9 is an exploded view of one bone engaging interface 98. As shown, the bone engaging interface 98 is defined between a pair of adjacent threads 96 on the shaft 90 of the bone screw. For purposes of illustration and not limitation, the bone engaging interface is shown constructed similar to the bone engaging interface illustrated in FIGS. 2-3. That is, the bone engaging interface includes a plurality of inverse frustoconical protrusions 100 spaced from one another to define cavities (not numbered) that can be deposited with bone growth promoting material. The bone engaging interface 98 is shown as being formed on the shaft between adjacent threads 96. However, it is contemplated that the threads themselves may also be laser machined to have a bone engaging interface such as that described herein.

The present disclosure has been described with respect to a representative intervertebral prosthetic joint and a representative bone screw; however, the present disclosure is applicable with other implants not specifically described herein. For example, the present disclosure is also applicable with bone plates, cages, and artificial discs. The present disclosure is also applicable with knee, hip, and other anatomical implants in addition to the vertebral implants described herein.

As described herein, the bone engaging interfaces are preferably formed using laser machining. With laser machining the size, shape, orientation, position, depth, and pattern of the bone engaging interfaces can be controlled. In a preferred example, the cavities defined in the bone engaging interfaces have a depth of approximately 100 microns; however, the present disclosure is not so limited. Also, while laser machining has been identified as one technique for engineering the surfaces of an implant, it is recognized that other techniques, such as EDM, could be used for engineering the surfaces of an implant.

Although only a few exemplary embodiments have been described in detail above, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top,” “upper,” “lower,” “bottom,” “left,” “right,” “cephalad,” “caudal,” “upper,” and “lower,” are for illustrative purposes only and can be varied within the scope of the disclosure. Further, the embodiments of the present disclosure may be adapted to work singly or in combination over multiple spinal levels and vertebral motion segments. Also, though the embodiments have been described with respect to the spine and, more particularly, to vertebral motion segments, the present disclosure has similar application to other motion segments and parts of the body. In the claims, means-plus-function clauses are intended to cover the elements described herein as performing the recited function and not only structural equivalents, but also equivalent elements.

We claim:

1. An implant comprising:
a body; and
a bone engaging interface formed on a portion of the body and shaped to favor movement of the implant in a first direction and to resist movement in a second direction opposite the first direction.

2. The implant of claim 1 wherein the bone engaging interface includes a set of protrusions defining a plurality of bone ingrowth cavities.

3. The implant of claim 2 wherein the set of protrusions are angled in the second direction relative to the body.

4. The implant of claim 2 wherein the set of protrusions includes a first subset of protrusions and a second subset of protrusions, the first subset of protrusions extending from the body more than the second subset of protrusions.

5. The implant of claim 2 further comprising bone growing material in the plurality of bone ingrowth cavities.

6. The implant of claim 1 wherein the bone engaging interface is configured to resist movement in the second direction when a load is placed thereon.

7. The implant of claim 1 wherein the bone engaging interface is formed by laser machining the body.

8. A intervertebral prosthetic joint comprising:
a first articular component and a second articular component;
a first bone engaging surface defined on a portion of the first articulur component;
a second bone engaging surface defined on a portion of the second articulur component, and
wherein each bone engaging surface is configured to provide a migration promoting interface along a first direction and provide an anti-migratory interface along a second direction opposite the first direction.

9. The joint of claim 8 wherein each bone engaging surface includes a laser-machined set of protrusions defining a first plurality and a second plurality of bone ingrowth cavities.

10. The joint of claim 9 wherein at least a portion of the first plurality and the second plurality of bone ingrowth cavities contains bone growth promoting material.

11. An implant comprising:
an implant body having a bone engaging surface;
a plurality of cavities formed by machining a portion of the bone engaging surface of the implant body; and
bone growth material deposited in the plurality of cavities.

12. The implant of claim 11 further comprising a set of protrusions extending from the implant body and defining the plurality of cavities, the set of protrusions being directionally aligned at an angle relative to the implant body.

13. The implant of claim 12 wherein the set of protrusions provide a non-gripping interface when the implant body is translated along a first direction and provide a bone-gripping interface when the implant body is translated along a second direction opposite the first direction.

14. The implant of claim 12 wherein the set of protrusions includes a first subset of protrusions and a second subset of protrusions different in at least one of height, spacing, shape, and depth from the first subset of protrusions.

15. A surgical method for positioning an intervertebral implant, the method comprising:
preparing a disc space for reception of an intervertebral implant;
inserting the intervertebral implant, along a first direction, into the disc space;
commencing withdrawing the intervertebral implant from the disc space along a second direction opposite the first direction; and
terminating withdrawal of the intervertebral implant when the intervertebral implant engages a vertebral body defining the disc space.

16. A method of manufacturing an implant, the method comprising:
forming an implant body;
machining the implant body to define a plurality of protrusions and cavities; and
depositing bone growth promoting material in the cavities.

17. The method of claim 16 wherein the machining includes defining the protrusions to be angled in a direction of implantation.

18. The method of claim 16 wherein the defining includes forming the protrusions to provide a non-gripping face for when the intervertebral implant is inserted into a disc space and to provide a gripping face for when the intervertebral implant is withdrawn from the disc space.

19. The method of claim 16 wherein the machining includes defining a first set of protrusions to have a first height and a second set of protrusions to have a second height different from the first height.

20. The method of claim 16 wherein the machining includes laser machining.

21. The method of claim 16 wherein the machining includes EDM.

22. A bone screw comprising:
a shaft;
a plurality of threads formed on the shaft and spaced from one another; and
a bone ingrowth cavity formed in the shaft in a space between a pair of threads.

23. The bone screw of claim 22 wherein the bone ingrowth cavity is formed by laser-machining of the shaft.

24. The bone screw of claim 22 further comprising bone growth material deposited in the bone ingrowth cavity.

25. The bone screw of claim 22 wherein the bone ingrowth cavity is shaped to have an opening that is smaller in width than a maximum width of the bone ingrowth cavity.
